(19) United States
(12) Patent Application Publication
(10) Pub. No.: US 2014/0358120 A1
(43) Pub. Date: Dec. 4, 2014

(54) MEDICAL SYSTEM AND CATHETER CONNECTOR APPARATUS

(71) Applicants: Brett Haarala, Framingham, MA (US); Richard Braga, Taunton, MA (US)

(72) Inventors: Brett Haarala, Framingham, MA (US); Richard Braga, Taunton, MA (US)

(21) Appl. No.: 13/908,145

(22) Filed: Jun. 3, 2013

(51) Int. Cl. A61M 39/10

(52) U.S. Cl. CPC A61M 39/10; A61M 39/105
USPC 604/508; 604/533

(57) ABSTRACT

A medical system for connecting a multiple lumen medical catheter to associated medical instrumentation includes a connector member defining a longitudinal axis and having a first end for releasable coupling to the medical catheter and a second end for coupling to medical instrumentation. A mounting segment is adjacent the first end of the connector member. The mounting segment includes first and second mounting elements extending in a general longitudinal direction and arranged in spaced relation, and being adapted for reception within respective lumens of the catheter. The first and second mounting elements define inner opposing surfaces. At least one of the opposing surfaces may have an irregular surface portion dimensioned to engage the septum wall of the catheter to facilitate gripping engagement with the septum wall to assist in coupling of the connector member with the catheter.

### BACKGROUND

1. Technical Field

The present disclosure relates generally to a medical system, and, more particularly, relates to a medical system incorporating a catheter connector apparatus.

2. Description of the Related Art

Catheters are flexible medical instruments intended for the withdrawal and introduction of fluids relative to body cavities, ducts, and vessels. Catheters have particular application in hemodialysis procedures where blood is withdrawn from a blood vessel for treatment, and subsequently returned to the blood vessel for circulation. Known hemodialysis catheters include multiple lumens, such as dual lumen or triple-lumen catheters, permitting bi-directional fluid flow within the catheter whereby one lumen is dedicated for withdrawal of blood and the other lumen is dedicated for returning the treated blood to the vessel. During an exemplary hemodialysis procedure, a multiple lumen catheter is inserted into a body and blood is withdrawn through an arterial lumen of the catheter. The removed blood is directed to a hemodialysis unit which dialyzes, or purifies, the blood to remove waste, and toxins. The dialyzed blood is returned to the subject through a venous lumen of the catheter.

Various devices are employed for the insertion of hemodialysis catheters including, e.g., tunnelers, introduction stylets or the like. A known technique of inserting a catheter includes forming a subcutaneous tunnel between two spaced openings in the skin with the use of a trocar or the like. The catheter end is attached to the insertion stylet or trocar and pulled though the tunnel to expose the catheter which is subsequently inserted into, e.g., the jugular vein or other vessel, and routed to the heart. The catheter end must be secured to the trocar in a manner which prevents detachment during passage through the tissue. In addition, the profile of the insertion devices and catheter may need to be minimized for ease of passage through the subcutaneous tissue. Adaptability of a broad range of catheters, tunnelers and sheaths is also a consideration.

### SUMMARY

Accordingly, the present disclosure is directed to a medical system for connecting a medical catheter to associated medical instrumentation. The medical system includes a connector member defining a longitudinal axis and having a first end for connecting to the medical catheter and a second end for coupling to medical instrumentation. A mounting segment is disposed adjacent the first end of the connector member. The mounting segment includes at least one mounting element extending in a general longitudinal direction, and being adapted for reception within a lumen of the catheter. The at least one mounting elements has a contacting surface dimensioned to engage a wall of the catheter to facilitate gripping engagement with the wall to assist in coupling of the connector member with the catheter. The contacting surface may define an irregular surface portion dimensioned to facilitate coupling of the mounting element to the wall of the catheter. The irregular surface portion may have at least one of a projecting member and a recess.

In an alternate embodiment, the medical system is adapted for connecting a multi-lumen medical catheter to associated medical instrumentation. The system includes a connector member defining a longitudinal axis and having a first end for coupling to the medical catheter and a second end for coupling to medical instrumentation and a mounting segment adjacent the first end of the connector member. The mounting segment includes first and second mounting elements extending in a general longitudinal direction and arranged in spaced relation, and being adapted for reception within respective lumens of the catheter. The first and second mounting elements define inner opposing surfaces. At least one inner opposing surface is dimensioned to engage a septum wall of the catheter to facilitate gripping engagement with the septum wall to assist in coupling of the connector member with the catheter. Each opposing surface may be dimensioned to engage the septum wall of the catheter, and to establish an interference relation with the septum wall to releasably mount the catheter to the connector member.

The at least one opposing surface may define an irregular surface portion. The irregular surface portion may have at least one projecting member. The at least one projecting member includes a leading surface extending at a first angle with respect to the longitudinal axis and arranged to facilitate insertion within a respective lumen of the catheter and a trailing surface extending at a second angle with respect to the longitudinal axis and arranged to inhibit removal from the respective lumen. The first angle of the leading surface is an oblique angle and is less than the second angle of the trailing surface. Each opposing surface of the first and second mounting elements may include the irregular surface portion having the at least one projecting member. The irregular surface portion of the at least one opposing surface may include a plurality of recesses arranged in longitudinally spaced relation.

In an alternate embodiment, the at least one opposing surface may define an irregular surface portion having an undulating arrangement. The undulating arrangement may include a plurality of alternating peaks and valleys. Each opposing surface of the first and second elements may define the irregular surface portion having the undulating arrangement. The opposing surfaces of the first and second mounting elements may be arranged as mating surfaces.

The medical system also may include a cover releasably mountable to the first end of the connector member in the absence of the medical catheter, to thereby substantially enclose the mounting segment. The cover may include an inner dividing wall separating at least two internal lumens. The first and second mounting elements of the mounting segment are adapted for reception within respective lumens of the cover whereby the opposing surfaces of the first and second mounting elements are dimensioned to engage the dividing wall of the cover to couple the connector member and the cover. At least one or both opposing surfaces of the first and second elements may include an irregular surface portion dimensioned to facilitate gripping engagement with the dividing wall to assist in coupling the connector member and the cover.

The medical system may include a second mounting segment adjacent the second end of the connector member. The second mounting segment has the first and second mounting elements extending in a general longitudinal direction and arranged in spaced relation, and being adapted for reception within respective lumens of the catheter, the first and second mounting elements defining inner opposing surfaces. The opposing surfaces are dimensioned to engage a septum wall of a second catheter to couple the connector member and the second catheter to secure to catheters in series. At least one or each opposing surface of the first and second elements may include an irregular surface portion dimensioned to facilitate gripping engagement with the septum wall of the second catheter to assist in coupling the connector member and the second catheter.

The medical system may include an insertion stylet coupled to the second end of the connector member and a catheter which is releasably coupled to the first end of the connector member. The catheter includes a pair of lumens separated by a septum wall.

In another embodiment, a medical system for facilitating insertion of a dual lumen catheter through a subcutaneous tunnel, includes an insertion stylet defining a longitudinal axis and having leading and trailing ends and a mounting segment adjacent the leading end of the insertion stylet. The mounting segment includes first and second mounting elements extending in a general longitudinal direction and arranged in spaced relation, and being adapted for reception within respective lumens of the dual lumen catheter. The first and second elements define inner opposing surfaces with at least one, possibly, each, of the opposing surfaces having an irregular surface portion defining at least one recess or projection and dimensioned to contact a septum wall separating the lumens of the catheter to facilitate gripping engagement with the septum wall to assist in coupling of the connector member with the catheter. A cover may be releasably mountable to the leading end of the insertion stylet to substantially enclose the mounting segment.

The insertion stylet may include a handle adjacent the trailing end and a bent portion disposed between the handle and the mounting segment. The bent portion is dimensioned to displace the handle relative to the longitudinal axis in a general radial direction.

A method for implanting a catheter for use in a hemodialysis procedure is also disclosed. The method includes the steps of:

accessing the venous system of a patient with a catheter through a first opening in tissue, the catheter having at least two lumens separated by a septum wall;

forming, with a tunneler instrument, a subcutaneous tunnel between the first opening in tissue and a second opening in tissue;

connecting a connector apparatus to the tunneler instrument, the connector apparatus including a mounting segment defining first and second mounting elements extending in a general longitudinal direction and arranged in spaced relation, and being adapted for reception within respective lumens of the catheter, the first and second mounting elements defining inner opposing surfaces, at least one inner opposing surface dimensioned to engage the septum wall of the catheter to facilitate gripping engagement with the septum wall to assist in coupling of the connector member with a catheter end of the catheter; and

moving the tunneler instrument and the catheter end through the subcutaneous tunnel to expose the catheter end through one of the first and second openings in tissue; and

connecting the catheter end to a hemodialysis apparatus.

### BRIEF DESCRIPTION OF THE DRAWINGS

Preferred embodiments of the disclosure will be better understood with reference to the accompanying drawings wherein:

FIG. 1 is a perspective of a medical system illustrating an insertion stylet, a catheter and a connector apparatus for releasably coupling the catheter;

FIG. 2 is an enlarged perspective view of the area of detail indicated in FIG. 1 illustrating the bifurcated segment of the connector apparatus prior to coupling to the catheter;

FIG. 3 is a perspective view of the connector apparatus and a mounting portion of the insertion stylet;

FIG. 4 is a perspective view with portions cutaway illustrating the irregular internal surface of the mounting elements of the bifurcated segment;

FIG. 5 is a side cross-sectional view of the bifurcated segment of the connector apparatus;

FIG. 6 is a cross-sectional view taken along the lines 6-6 of FIG. 5;

FIG. 7 is a side cross-sectional view illustrating the bifurcated segment of the connector apparatus coupled to the catheter;

FIG. 8 is a cross-sectional view taken along the lines 8-8 of FIG. 7;

FIG. 9 is a perspective view of the medical system incorporating a dilator for mounting to the connector apparatus;

FIGS. 10-11 are each views of the chest area of the subject illustrating a methodology of use of the medical system in a reverse tunneling procedure;

FIG. 12 is a side plan view of an alternate embodiment of the medical system including an insertion stylet and integral connector apparatus, and a releasable cover;

FIG. 12A is a side plan view of an alternate embodiment the insertion stylet of the medical system of FIG. 12;

FIG. 13 is an enlarged perspective view of the area of detail indicated in FIG. 12 illustrating the releasable cover prior to coupling to the bifurcated segment of the connector apparatus;

FIG. 14 is a side cross-sectional view illustrating the connector apparatus of FIG. 12;

FIG. 15 is a side cross-sectional view illustrating the connector apparatus of FIG. 12 coupled to the releasable cover;

FIGS. 16-17 are each views of the chest area of the subject illustrating a methodology of use of the medical system of FIG. 12 in a reverse tunneling procedure;

FIG. 18 is a perspective view illustrating an alternate embodiment of a cover for use with the medical system;

FIG. 19 is an enlarged view of the area of detail identified in FIG. 18;

FIG. 20 is a side cross-sectional view illustrating the cover mounted to the connector apparatus;

FIG. 21 is a side plan view of another alternate embodiment of a connector apparatus;

FIG. 22 is a side cross-sectional view illustrating the connector apparatus of FIG. 21 releasably coupled to the catheter;

FIG. 23 is a perspective view of an alternate embodiment of the connector apparatus including opposed bifurcated segments for connecting two catheters in series;

FIG. 24 is a perspective view of an alternate embodiment of the connector apparatus incorporating three mounting elements for use with a triple lumen catheter;

FIG. 25 is an axial view of the connector apparatus of FIG. 24;

FIG. 26 is a perspective view of an alternate embodiment of the connector apparatus incorporating a single mounting element for use with a single lumen catheter;

### DETAILED DESCRIPTION OF THE EMBODIMENTS

The exemplary embodiments of the present disclosure are directed to a medical system incorporating a catheter connector apparatus adapted to connect a catheter to a variety of instruments during the course of a surgical procedure. The connector apparatus of the present disclosure has particular application for use in positioning a catheter during a hemodialysis catheter implantation procedure. It is envisioned, however, that the present disclosure may be employed with a range of applications including surgical, diagnostic and related treatments of diseases and body ailments of a subject.

In the discussion that follows, the term “proximal” or “trailing” will refer to the portion of a structure that is closer to a clinician, while the term “distal” or “leading” will refer to the portion that is further from the clinician. As used herein, the term “subject” refers to a human patient or other animal. The term “clinician” refers to a doctor, nurse or other care provider and may include support personnel.

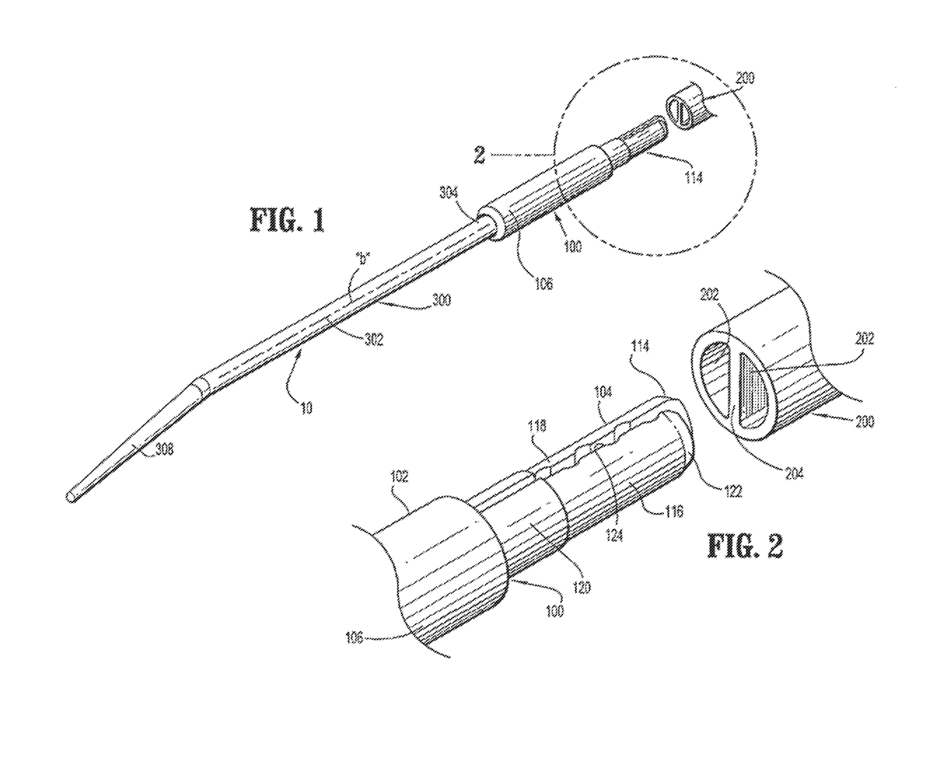

Referring now to the drawings wherein like components are designated by like reference numerals throughout the several views, FIGS. 1-4 illustrate the system 10 incorporating a connector apparatus 100 for connecting a medical catheter 200 to associated instrumentation, e.g., such as an insertion stylet 300, in accordance with the principles of the present disclosure. Connector apparatus 100 may be integrally formed with insertion stylet 300, or, in one embodiment, may be releasably couplable to the insertion stylet 300. System 10 may be utilized with system 10 for placement of catheter 200 through ante-grade and reverse tunneling methods such as the methods disclosed in U.S. Pat. No. 5,509,897 to Twardowski.

Connector apparatus 100 includes connector member 102 having first end 104 for releasable coupling to catheter 200 and second end 106 for connection to tunneling instrument 300. Connector member 102 defines longitudinal axis “k”. Second end 106 includes cylindrical segment 110 having internal thread 112 extending at least partially along the length of the cylindrical segment 110. Internal thread 112 assists in releasably connecting connector member 102 to insertion stylet 300.

With reference now to FIGS. 2-6, first end 104 of connector member 102 incorporates bifurcated mounting segment 114. Bifurcated segment 114 includes first and second mounting elements 116, 118 extending in a general longitudinal direction relative to longitudinal axis “k”. First and second mounting elements 116,118 are dimensioned to be received within respective lumens of catheter 200, e.g., a dual lumen catheter, as will be discussed hereinbelow. First mounting element 116 defines a longitudinal length which is less than second mounting element 118 to define a staggered arrangement as shown. This staggered length may facilitate insertion of first and second mounting elements 116, 118 within the lumens of catheter 200. In the alternative, first and second elements 116, 118 may be substantially identical in length. First and second elements 116, 118 define bifurcated collar 120 adjacent second end 106 of connector member 102 and tapered end surfaces 122 opposing the collar 120. Bifurcated collar 120 defines a cross-sectional dimension or diameter which is slightly larger than the corresponding cross-sectional dimensions of the remaining portions of first and second mounting elements 116,118. Tapered end surfaces 122 assist in initial positioning of first and second mounting elements 116, 118 within the lumens of catheter 200.

First and second mounting elements 116, 118 are spaced a predetermined distance to define slot 124 between the first and second elements 116, 118. Each of first and second elements 116, 118 define internal surface 126 adjacent the slot 124 and in diametrical opposed relation. Internal surfaces 126 may define irregularities which may further assist in mounting of connector member 102 to catheter 200. The term “irregularities” or “irregular surface” is to be interpreted as being inclusive of, but, not limited to, any surface having projections, barbs, recesses, protrusions, coatings or the like, or any surface altered or modified during or subsequent to manufacture of the instrument, which facilitates or enhances contact and/or engagement with another surface, e.g., a catheter surface or septum wall. In one embodiment, the irregularities incorporate a plurality of spaced recesses or concavities 128 in each internal surface 126. Recesses 128 may be arranged to intersect longitudinal axis “k” and may be arranged about an axis “m” in transverse relation to the longitudinal axis “k”. Adjacent recesses 128 of each of first and second elements 116, 118 may be separated by lands 130 to thereby define an alternating arrangement of lands 130 and recesses 128 for each of the first and second elements 116, 118 as shown.

Each land 130 may include projecting member 132 extending radially outwardly with respect to longitudinal axis “k”. Each projecting member 132 defines leading surface 134 and trailing surface 136. In one embodiment, leading surface 134 defines a first angle “α” with respect to longitudinal axis “k” and trailing surface 136 defines a second angle “θ” with respect to the longitudinal axis. First angle “α” of leading surface 134 is less than second angle “θ” of trailing surface 136 to assist in insertion of first and second elements 116, 118 within catheter 200. Trailing surface 136 arranged at second angle “θ” is adapted to engage an internal septum wall of catheter 200 in secured relation. First angle “α” may range from about 10 to about 60 degrees and second angle “θ” may range from about 60 degrees to about 90 degrees. Other arrangements are also envisioned. Multiple projections 132 extending from each land 130 are also envisioned.

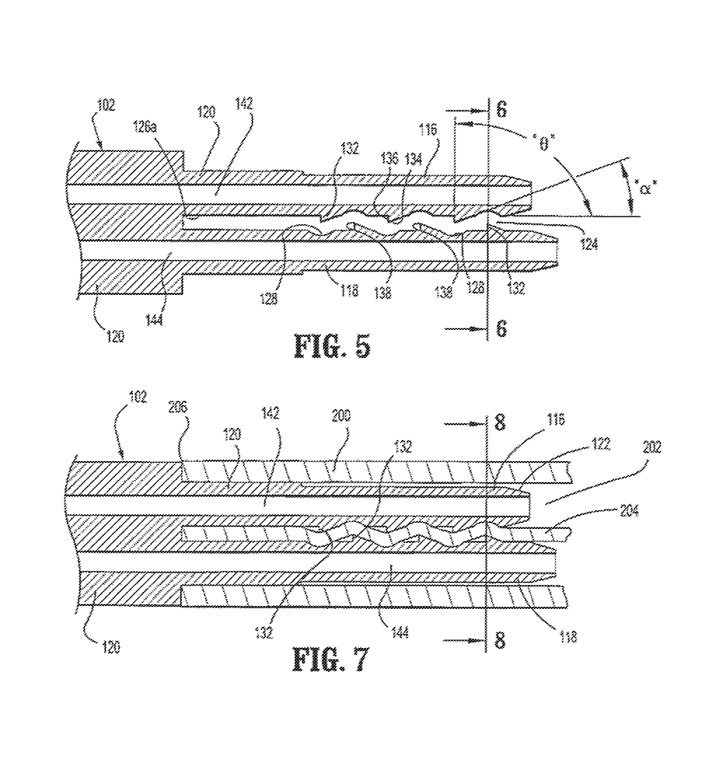

As best depicted in FIG. 5, in one arrangement, the apexes 136 of projecting members 132 of one of the first and second elements 116, 118 are in opposed relation to respective recesses 128 of the other of the first and second elements 116,118. This arrangement of internal surfaces 126 of first and second mounting elements 116, 118 defines a tortuous path adjacent slot 124, which assists in coupling and retention of catheter 200 to connector apparatus 100.

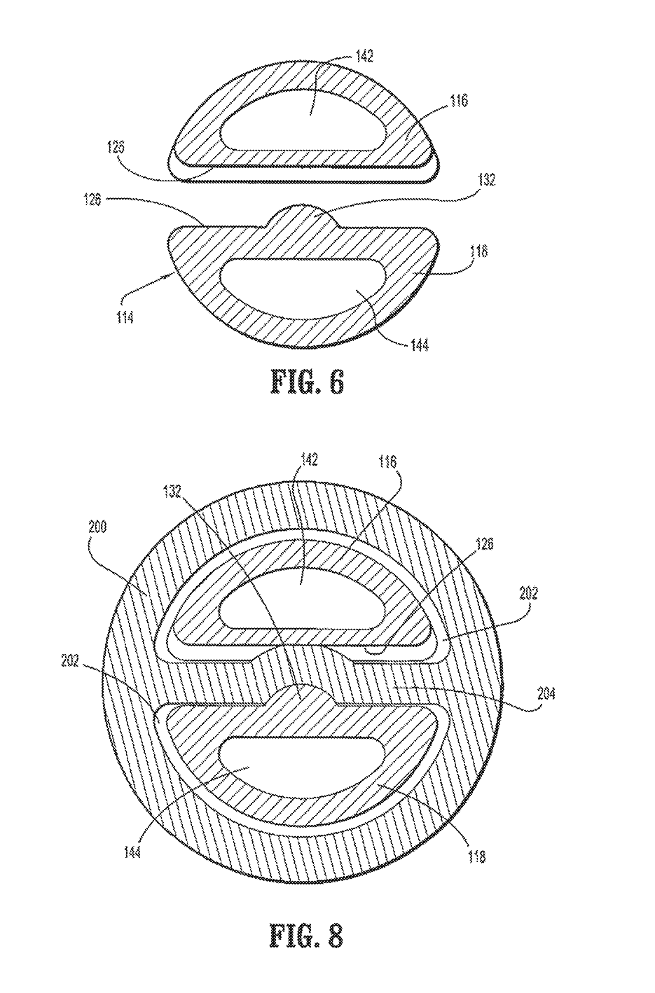

Referring to FIGS. 5 and 6, first and second mounting segments 116, 118 may each include respective axial lumens 142, 144 extending the lengths of the mounting segments 116, 118. Axial lumens 142, 144 may be in fluid communication with the interior of cylindrical segment 110 of connector member 102. Axial lumens 142, 144 may permit the passage of fluids for, e.g., priming of the catheter or distribution of fluids, medicants or the like. Alternatively, first and second mounting segments 116, 118 may be devoid of axial lumens 142, 144.

Referring now to FIGS. 1, 2 and 6, catheter 200 will be discussed. Catheters including single or multiple lumen catheters are well known in the art. In one embodiment, catheter 200 may be a dual lumen catheter including two generally D-shaped lumens 202 separated by septum wall 204. For example, suitable catheters include the Mahurkar® dual and triple lumen catheters available from Tyco Healthcare Group LP. Other catheters are also envisioned including triple lumen catheters, coaxial lumen catheters or any other suitable catheter having application in the removal and return of fluids to and from the subject in, e.g., a hemodialysis procedure. Catheter 200 may be fabricated from a suitable elastomeric, thermoplastic or polymeric material, and manufacturing through known extrusion or molding techniques or any other conventionally acceptable methodology. Catheter 200 is relatively flexible and may be capable of some deformation during, e.g., connecting of connector apparatus 100 to the catheter 200.

With reference to FIGS. 7-8, the coupling of connector apparatus 100 to catheter 200 will be discussed. Initially, tapered end surfaces 122 of first and second mounting elements 116, 118 are positioned within respective lumens 202 of catheter 200, and septum 204 is introduced within slot 124 defined between the first and second mounting elements 116, 118. Thereafter, first and second mounting elements 116, 118 are advanced relative to catheter 200 into lumens 202 of the catheter 200 until catheter end 206 contacts or “bottoms out” on collar 120 as best shown in FIG. 7. The oblique arrangement of leading surfaces 134 of projecting members 132 permit advancing movement of first and second elements 116, 118 within lumens 202 of catheter 200. In this secured position of catheter 200, the irregularities of internal surfaces 126 including, recesses 128, lands 130 and projections 132, deform septum wall 204 of the catheter 200 creating a tortuous path or arrangement substantially impeding removal of catheter 200 from connector apparatus 200. Apexes 138 defined at the intersection of leading and trailing surfaces 134, 136 of each projecting element 132 also may engage or partially penetrate septum wall 204 to further secure catheter 200 to connector apparatus 100. Accordingly, catheter 200 is secured to connector apparatus 100 to permit manipulation and positioning of the catheter 200 with additional instrumentation, e.g., an insertion stylet 300, as will be discussed. Catheter 200 may be removed from connector apparatus 100 via opposed forces exerted on each of the connector apparatus 100 and the catheter 200. However, the opposed forces required to release the components should be greater than any collective force exerted on the components during a surgical procedure to avoid inopportune release of the catheter 200 during, e.g., a tunneling procedure. In the alternative, the coupling of catheter 200 to connector apparatus 100 may be relatively permanent whereby the catheter end adjacent the connector apparatus 100 must be severed or cut to detach the connector apparatus 100.

It is further envisioned that internal surfaces 126 of first and second mounting elements 116,118 may be devoid of irregularities. For example, internal surfaces 126 may be substantially flat or planar along a substantial portion of the length of the internal surfaces 126. In FIG. 5, internal surfaces 126 are shown as having a planar component 126a adjacent collar 120. In accordance with this embodiment, internal surfaces 126 may be planar through at least a major portion of the length of the respective mounting element 116, 118. The internal surfaces 126 and slot 124 therebetween may be dimensioned to establish a friction fit or interference coupling with septum wall 204. Various coatings (e.g., an elastomeric coating) having physical characteristics to enhance a mechanical or frictional coupling are also envisioned to facilitate coupling of internal surfaces 126 to septum wall 204 of catheter 200.

As will be discussed in detail hereinbelow, this internal locking mechanism effected through mounting elements 116, 118 of connector apparatus 100 and catheter 200 reduces the overall profile of connector apparatus 100 to facilitate subsequent positioning of attached catheter 200 through a subcutaneous tunnel.

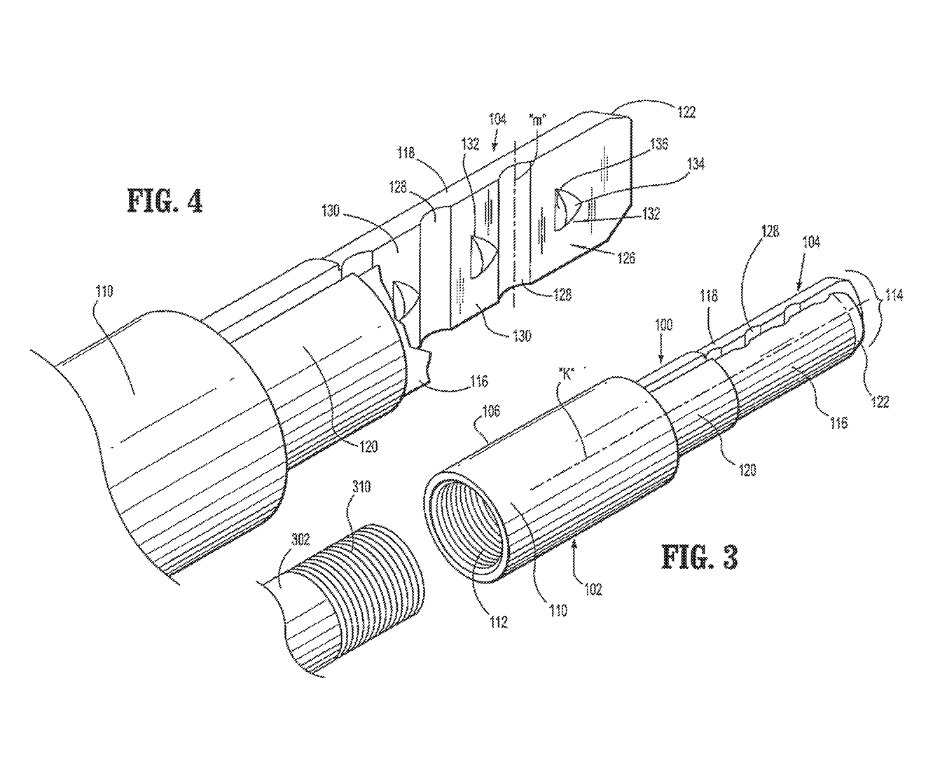

Referring again to FIGS. 1 and 3, insertion or tunneling stylet 300 of system 10 will be discussed. Insertion stylet 300 includes stylet body 302 defining longitudinal axis “b” and trailing and leading ends 304, 306, respectively. Stylet body 302 may be fabricated from a relatively flexible material including a biocompatible metal such as stainless steel or titanium or a suitable polymeric material. Stylet body 302 may be adapted to bend during advancement through the subcutaneous tissue. Stylet body 302 may incorporate various indicia or gradation markings (not shown) along its length to assist the clinician in determining the depth of insertion of the stylet body 302. Gradation markings may also consist of various radiopaque markings which are detectable during an x-ray scanning procedure.

Leading end 306 may include offset segment 308 which is dimensioned to facilitate passage through the subcutaneous tunnel formed in the tissue. Offset segment 308 may incorporate any angular or arcuate arrangement suitable to facilitate insertion and/or passage through the tissue when the insertion stylet 300 is manipulated by the clinician. In one embodiment, offset segment 308 is obliquely arranged with respect to longitudinal axis “b” of stylet body 302 at a relatively small angle. Other arrangements and angular relationships of offset segment 308 are also envisioned.

With continued reference to FIG. 3, insertion stylet 300 includes threaded segment 310 adjacent trailing end 304. Threaded segment 310 is adapted to cooperate with internal thread 112 of connector apparatus 100 to releasably connect the insertion stylet 300 and the connector apparatus 100. Other means to facilitate the releasable connection of the components are also envisioned including, but, not limited to, a bayonet coupling, snap coupling, interference fit, cam lock arrangements or any other coupling arrangement envisioned by one skilled in the art. In the alternative, insertion stylet 300 and connector apparatus 100 may be formed integrally as a single unit.

As indicated hereinabove, system 10 including connector apparatus 100, catheter 200 and insertion stylet 300, has application in a tunneling procedure for placement of the catheter 200 during hemodialysis of the subject. Generally stated, with connector apparatus 100 connected to insertion stylet 300 and catheter 200 releasably coupled to the connector apparatus, the clinician may advance/retract the insertion stylet 300 to corresponding position the catheter 200 within a subcutaneous tunnel created in the subject, to thereby properly orient the catheter 200 relative to the heart and major vessels of the subject for hemodialysis. One preferred tunneling methodology will be discussed in greater detail hereinbelow.

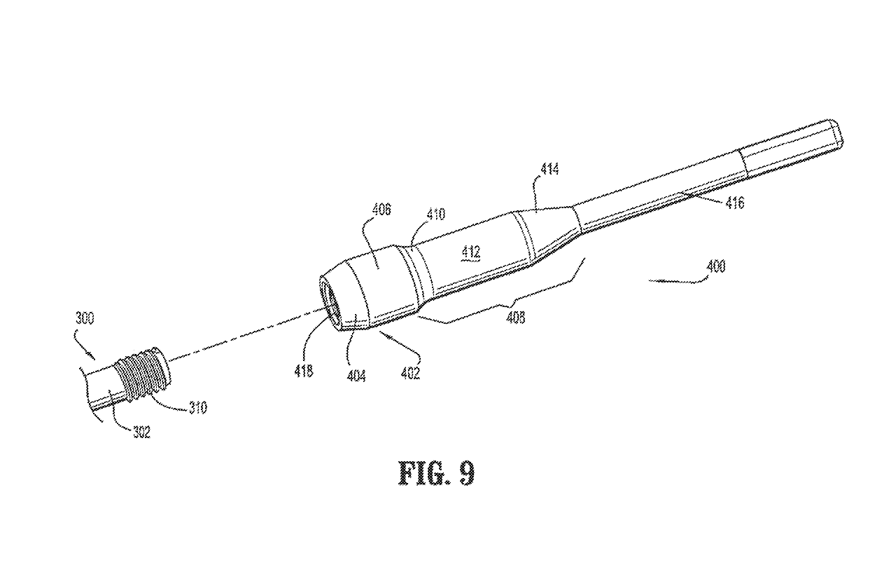

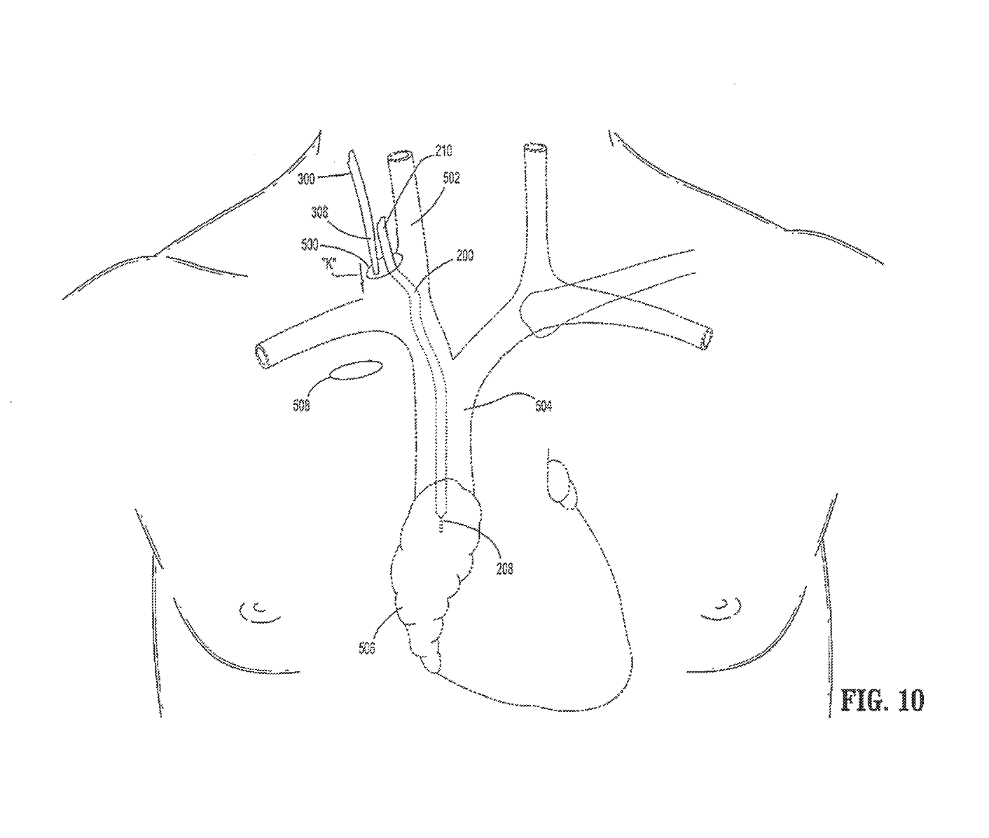

Referring now to FIG. 9, system 10 may further include dilator element 400. Dilator element 400 is adapted for releasable mounting to insertion stylet 300. Dilator element 400 may be incorporated to dilate a portion of the subcutaneous tunnel to, e.g., create an internal shelf within the subcutaneous tunnel to thereby facilitate placement of a cuff connected to the external surface of the catheter. Dilator element 400 includes first end segment 402 consisting of tapered segment 404 and cylindrical segment or collar 406. Dilator element 400 further includes intermediate segment 408 having tapered segment 410, cylindrical segment 412 and tapered segment 414. Dilator element 400 further includes second end segment 416 which is generally cylindrical in configuration. First end segment 402 may define an outer diameter or dimension which is greater than a corresponding outer diameter of insertion stylet 300.

Dilator element 400 may incorporate internal threads 418 which cooperate with external threads 310 of insertion stylet 300 to releasably connect the dilator element 400 to the insertion stylet 300. Other means for releasably connecting dilator element 400 to insertion style 300 are also envisioned including bayonet coupling, snap lock, frictional relationship of the like.

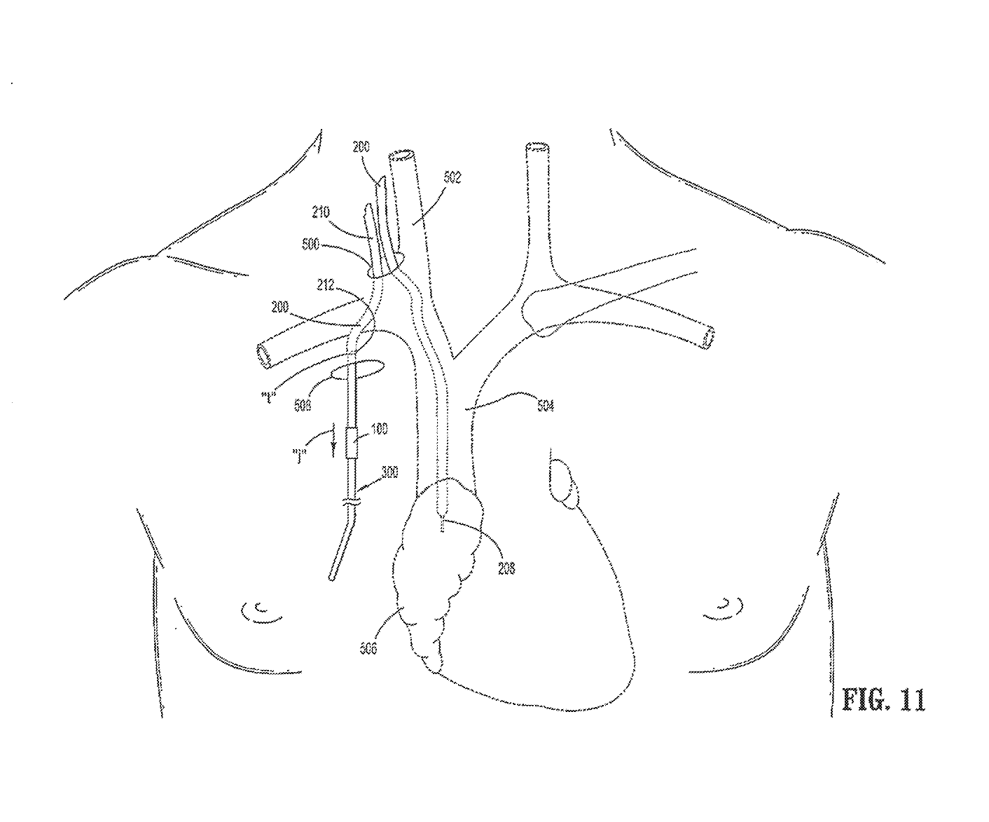

The use of system 10 will now be discussed in terms of a reverse tunneling procedure in connection with hemodialysis catheter placement. The application will be discussed in terms of creating a tunnel to deploy catheter 200 through the right jugular vein for positioning within the right atrium. As appreciated, the catheter 200 may be implanted in the right atrium via the left jugular vein, the right atrium through the right subclavian vein, the right atrium through the left subclavian vein, or implanted in the femoral vein of the subject. With reference to FIG. 10, an entry opening or venotomy 500 is made adjacent the midclavicular line, through the skin and the subcutaneous tissue. The internal jugular vein 502 is punctured using known techniques and the leading end 208 of catheter 200 is inserted through the internal jugular vein 502, the superior vena cavity 504 and into the right atrium 506. A guide wire may be positioned to access the heart to facilitate insertion of the leading end 208 of catheter 200 within the heart through techniques known in the art. The positioning of the leading end 208 of catheter 200 may be confirmed with an x-ray if desired. The trailing end 210 of the catheter may extend from the venotomy site 500.

Once the leading end 208 of catheter 200 is in position, attention is directed to preparing the subcutaneous tunnel incorporating the tunneling approach from the venotomy site 500 to an exit opening 508. Exit opening 508 is made adjacent to the chest wall below the venotomy site 500 to define one base of the tunnel. Thereafter, dilator element 400 is optionally mounted to insertion stylet 300. Offset segment 308 of insertion stylet 300 may be introduced within the venotomy site 500 and advanced toward the exit opening 508. As insertion stylet 300 is toward the exit opening 508, dilator element 400 engages internal tissue beneath the venotomy site 500. An enlarged tissue tract is thereby made to create a shelf for accommodating a cuff 212 (FIG. 11) positioned about the catheter 200 by advancing dilator element 400 a predetermined distance through the venotomy site 500 toward the exit opening 508. Segments 410,412,414 cooperated to dilate the tissue to form the internal tissue shelf. The predetermined distance corresponds to the desired location of the cuff 210. Insertion stylet 300 is then retracted toward the venotomy site 500 and the dilator element 400 is removed from the insertion stylet 300.

Referring now to FIG. 11, connector apparatus 100 is then mounted to insertion stylet 300 through threaded cooperation of the threaded components. First and second mounting elements 116, 118 of bifurcated segment 114 are then positioned within lumens 202 of catheter 200 to secure catheter 200 to the insertion stylet 300. Projecting elements 132 and the alternating arrangement of concavities 128 and lands 130 of internal surfaces 126 of first and second mounting elements 116,118 effectively engage the internals surfaces defining the lumens 206 of the catheter 200 and septum wall 204 in a manner to securely connect the catheter 200 to bifurcated segment 114 (FIG. 7). Once the catheter 200 is secured, insertion stylet 300 is readvanced in the direction of directional arrow “j” from the venotomy site 500 through the exit opening 508 until the bifurcated segment 114 is exposed from the exit opening 508 to expose the ends of the catheter 200. The relatively small profile provided by the internal locking mechanism of the connector apparatus 100 facilitates passage of the connector apparatus 100 through tissue. The catheter 200 is removed from its mounting to insertion instrument 1000 and connected to a hemodialysis machine. In one embodiment, catheter 200 is released from its mounting to connector apparatus 100 by exerting a linear force on catheter 200. Alternatively, with a more permanent connection with connector apparatus 100, the catheter 200 is severed or cut adjacent the connector apparatus 100 to expose the catheter ends.

In an alternate method, immediately subsequent to placement of catheter leading end 208 within the targeted site, bifurcated segment 114 is secured to the other end of catheter 200. With this arrangement, bifurcated segment 114 of connector apparatus 100 seals the catheter end and prevents leaking of blood, and permits aspiration of air through, e.g., axial lumens 142,144 in mounting segments 116,118.

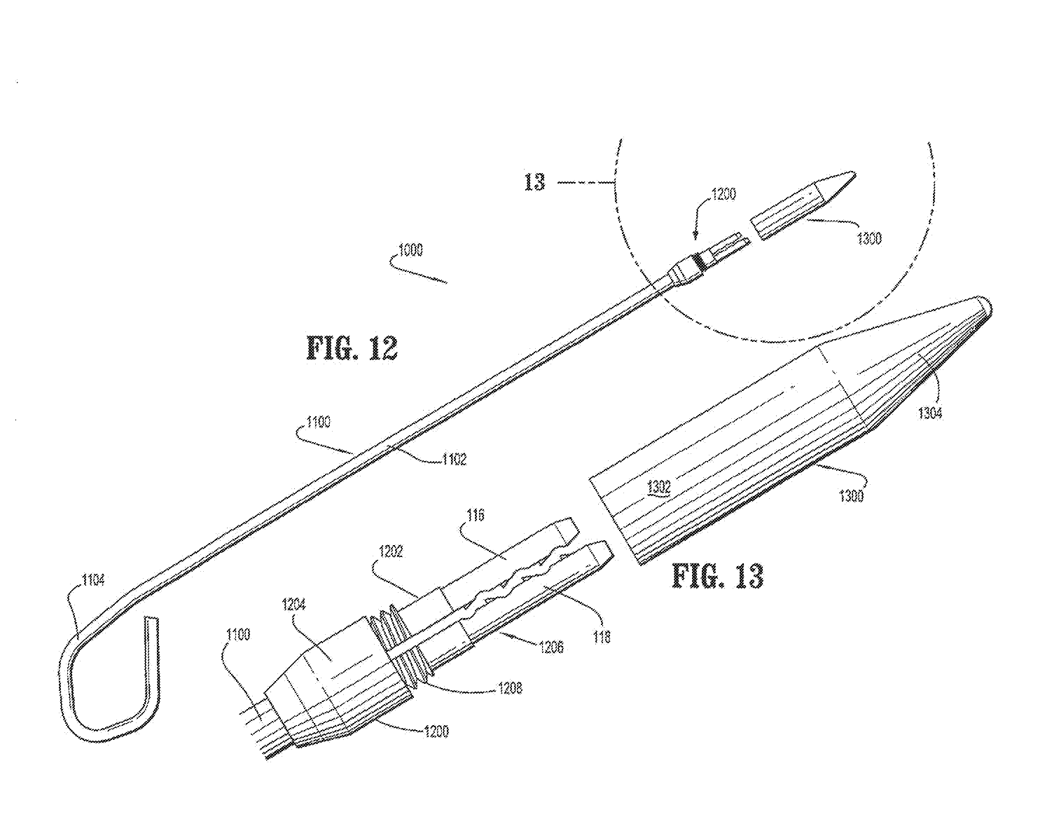

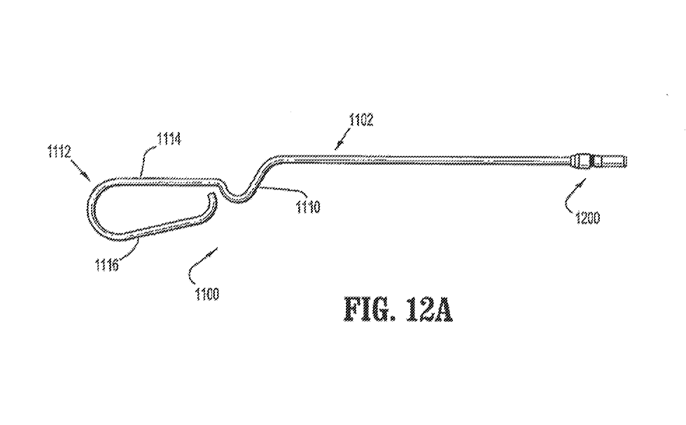

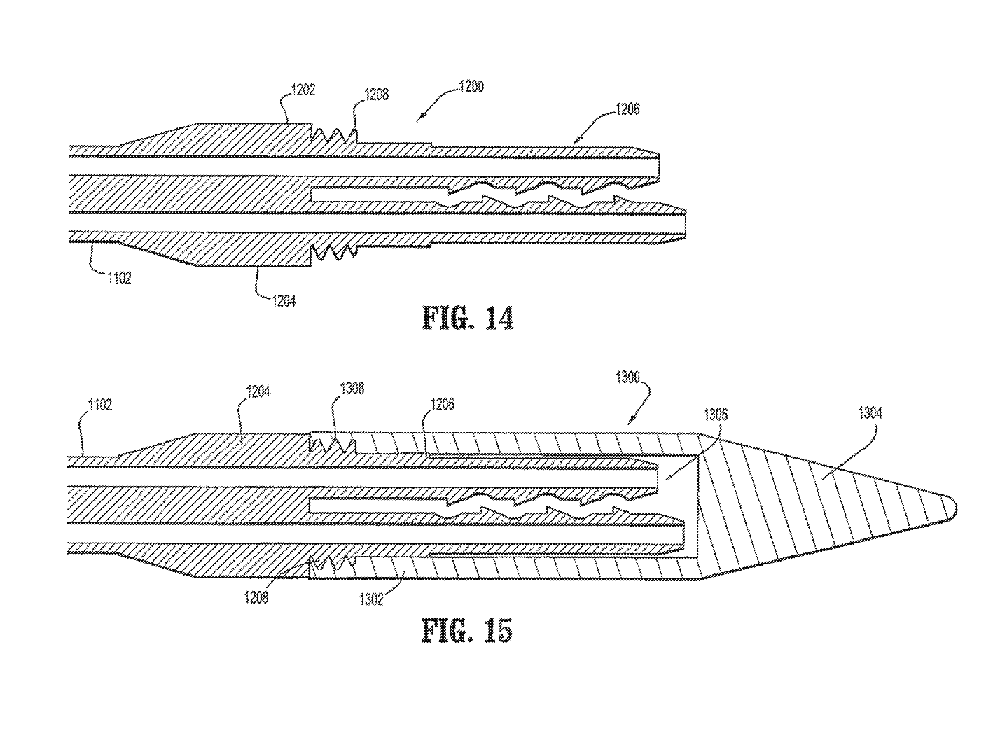

FIGS. 12-15 illustrate an alternate medical system 1000 in accordance with the present disclosure. Medical system 1000 includes insertion stylet 1100, connector apparatus 1200 and cover 1300. Insertion stylet 1100 includes stylet body 1102 and handle 1104 disposed at one end of the stylet body 1102. Handle 1104 may define at least a partial loop adapted for reception of, e.g., the clinicians' fingers. Connector apparatus 1200 is integrally formed with stylet body 1102 and is, thus, non-detachable from the stylet body 1102. Connector apparatus 1200 includes connector body 1202 having collar 1204 at one end and bifurcated segment 1206 at the other end. Bifurcated segment 1206 includes a threaded portion or series of threads 1208 adjacent collar 1206, to assist in releasably coupling cover 1300 to connector apparatus 1200. Bifurcated segment 1206 is substantially similar to bifurcated segment 114 of connector apparatus 100 discussed in connection with the embodiment of FIGS. 1-11. In an alternate embodiment of insertion stylet 1100 disclosed in FIG. 12A, stylet body 1102 includes generally “J” shaped bend 1110 adjacent handle 1112. J-shaped bend 1110 displaces handle 1112 relative to the remaining portion of stylet body 1102 whereby the clinician may place his hand on the upper portion or surface 1114 of handle 1112 in general longitudinal alignment with connector apparatus 1200 (with the fingers of the clinician within the loop defined by the handle 1112). This aligned position may facilitate advancement or retraction of insertion stylet 1100 relative to the tissue. As a further advantage, the displaced relation of handle 1112 relative to the remaining portion of stylet body 1102 will displace the clinician's hand from the chest area of the subject when, e.g., the clinician grasp's, with his/her palm, the lower surface 1116 of handle 1112 with the lower surface 1116 away from the chest area of the subject. Thus, the clinician's hand is not encumbered by the chest area of the subject and/or any other instrumentation extending from the chest. J-shaped bend 1110 also provides a surface upon which the clinician may grasp during manipulation of stylet body 1102. J-shaped bend 1110 may assume other configurations including a general U-shape, sinusoidal shape, arcuate shape, polygonal shape or other arrangements as appreciated by one skilled in the art. Handle 1112 is elongated relative to handle 1104 of the embodiment of FIG. 12.

Cover 1300 may be initially releasably coupled to connector apparatus 100 to assist in advancing insertion stylet 300 through tissue prior to connection to catheter 200. Cover 1300 may be fabricated from a biocompatible polymeric material or from a suitable elastomeric material capable of slight deformation when mounted to connector apparatus 200. Releasable cover 1300 includes trailing cylindrical segment 1302 and leading tapered segment 1304. Releasable cover 1300 defines single lumen 1306 within cylindrical segment 1302 of the releasable cover 1300. Cylindrical segment 1302 of releasable cover 1300 further includes internal thread 1308. Internal thread 1308 cooperates with external thread 1208 of bifurcated segment 1206 of connector body 102 to releasably secure cover 1300 to connector apparatus 1200.

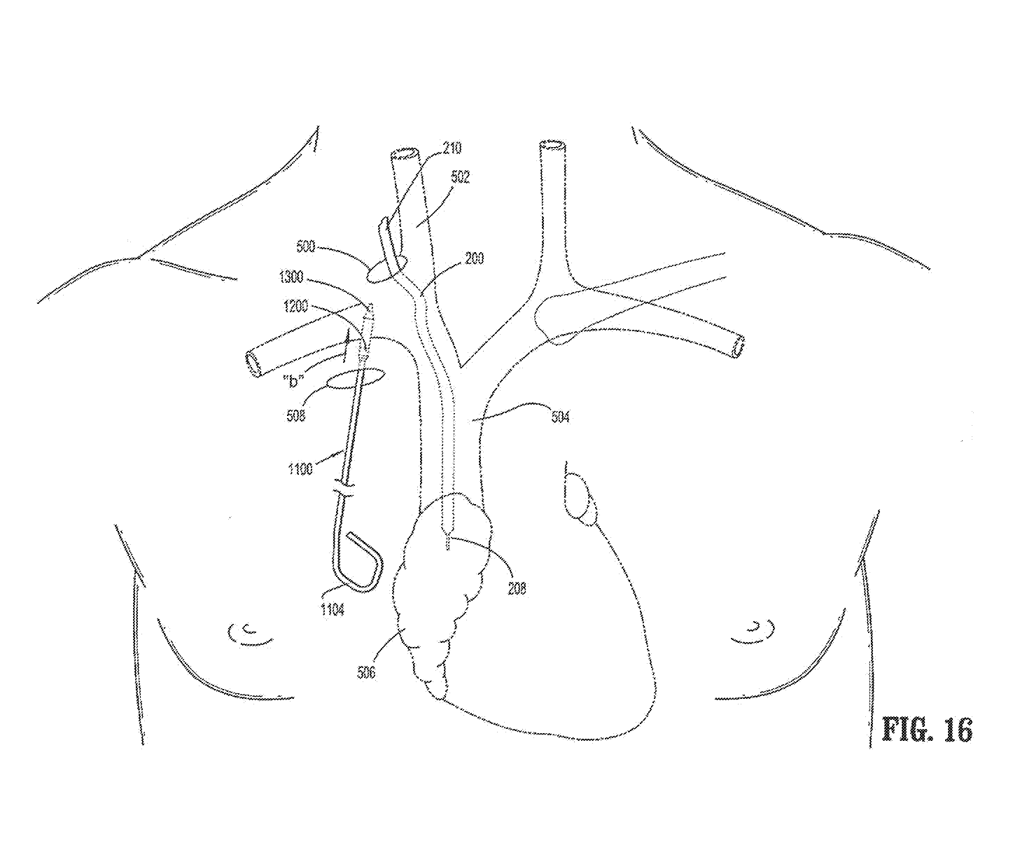

Insertion stylet 1100, connector apparatus 1200 with mounted cover 1300 may be used to tunnel or advance through the subcutaneous tunnel in accordance with, e.g., a revere tunneling procedure for catheter insertion. In one contemplated method, a tunnel may be made from the exit opening 508 to the venotomy site 500. Referring now to FIG. 16, insertion stylet 1100 with cover 1300 mounted thereto in the condition depicted in FIG. 15 is introduced within the exit site 508 and advanced toward the venotomy site 500 (as shown schematically by arrows “B”) to create or pass through a subcutaneous tunnel. Cover 1300 of insertion stylet or tunneling instrument 1100 is designed with optimized geometry to permit effective dissection of subcutaneous tissue as it is advanced toward the venotomy site 500. Once cover 1300 is exposed or extends from the venotomy site 500, the cover 1300 is removed from insertion stylet 1100. Thereafter, optionally, a dilator element 400 (such as disclosed in FIG. 9) may be releasably secured to insertion stylet 1100 via any of the aforementioned connection means (e.g., through threaded cooperation of external threads 1208 of insertion stylet 1100 and internal threads 418 of dilator element 400), and the insertion stylet 1100 is retracted or pulled back a predetermined distance within the created subcutaneous tunnel. This activity consequently causes a portion of the subcutaneous tunnel adjacent the venotomy site 500 to be dilated due to segments 410, 412, 414 of dilator element 400 engaging the internal tissue. The predetermined distance will generally correspond to the desired location of the cuff attached to the catheter 200. The juncture of the internal tissue dilated by dilator element 400 and the remaining portion of the subcutaneous tunnel defines a ledge or shelf to accommodate the cuff of the catheter. Once the shelf is created, insertion stylet 1100 is again advanced towards the venotomy site 500 to at least partially expose dilator element 400. Dilator element 400 is thereafter removed from insertion stylet 1100.

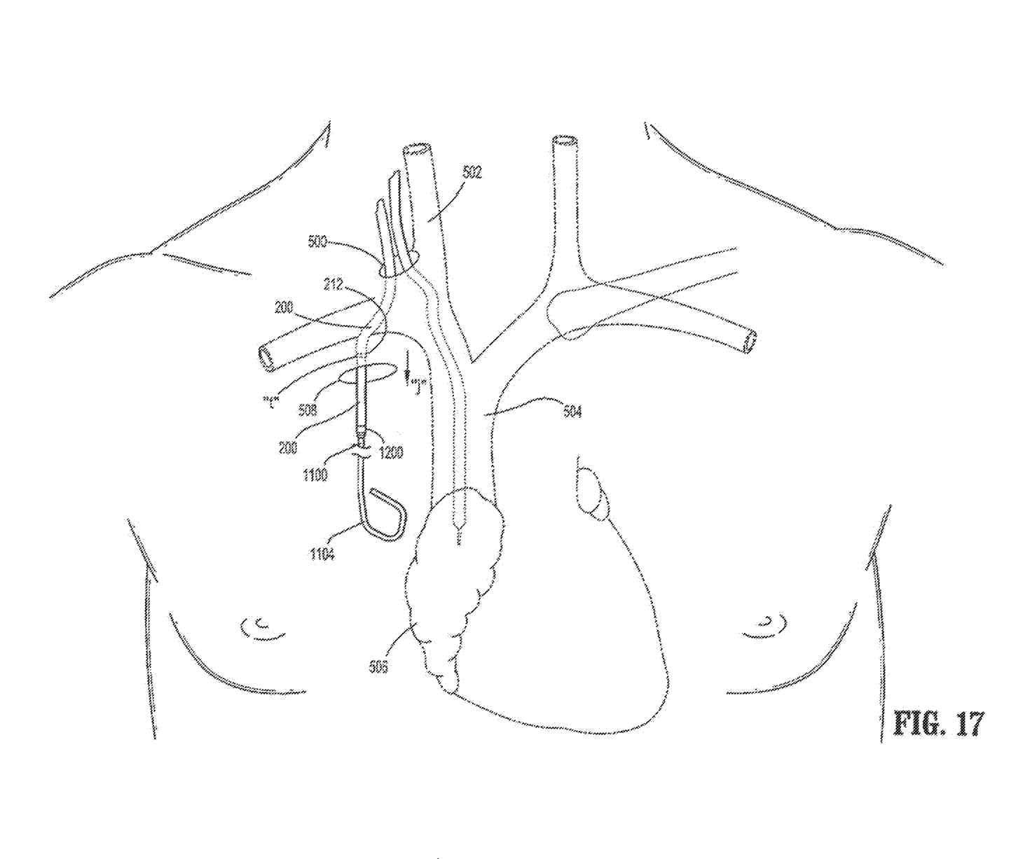

Referring now to FIG. 17, catheter 200 is then secured to insertion stylet 1100 by positioning first and second mounting elements 116,118 of bifurcated segment 114 within the lumens 206 of the catheter 200. With the catheter 200 attached, insertion stylet 1100 is drawn or pulled back toward the exit site 508 in the direction of directional arrows “j” as shown in FIG. 17. Once the trailing end of the catheter 200 is exposed from the exit site 508, the catheter 200 is released from insertion stylet 1100. The catheter 200 may then be connected to a hemodialysis machine. In FIG. 17, cuff 212 of catheter 200 is shown within the shelf “t” of the subcutaneous tunnel. The size of the shelf “t” is exaggerated for exemplative purposes.

As a further alternate, it is envisioned that connector apparatus 100 with bifurcated segment 114 may be releasably connectable to insertion stylet 1100 in a manner similar to that disclosed in connection with the embodiment of FIG. 1. A tip (not shown) may be connectable to insertion stylet 1100 during initial advancement of the insertion stylet 1100 from the exit opening 508 to the venotomy site 500. Dilator element 400 may be then optionally secured to insertion stylet 1100 after the tip is removed to foam the internal tissue ledge. Once the ledge is created, insertion stylet 1100 is advanced through the venotomy site 500 to remove dilator element 400 and attach the releasable connector apparatus 100 with bifurcated segment 114 and catheter 200 to the insertion stylet 1100. Insertion stylet 1100 may be retracted through exit site 508 in a similar manner to that described hereinabove.

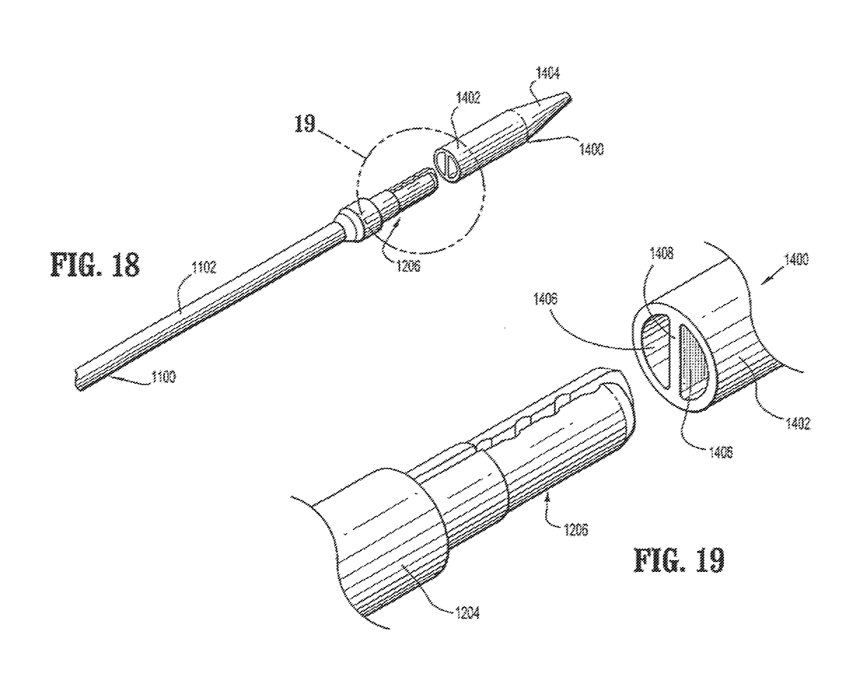

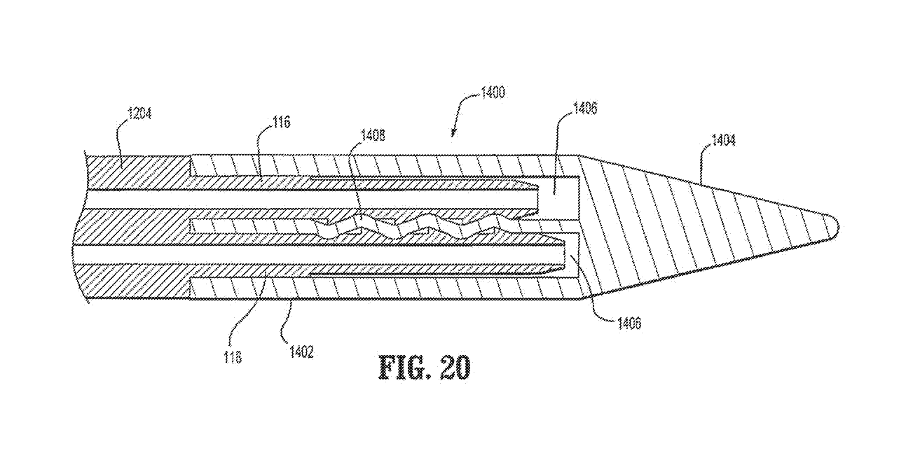

FIGS. 18-20 illustrate and alternate embodiment of cover for mounting to connector apparatus 100. Insertion stylet 1100 and connector apparatus 1200 may be similar to the embodiment described in connection with FIG. 12; however, connector apparatus is devoid of external threads 1208. Cover 1400 includes trailing section 1402 and leading section 1404. Trailing section 1402 is adapted to be positioned over bifurcated segment 114 of connector apparatus 100 to substantially enclose the bifurcated segment 114. Trailing section 1402 may be generally cylindrical in configuration. In one embodiment, trailing section 1402 of cover 1400 includes double lumens 1406 separated by dividing or septum wall 1408 similar in configuration to dual lumens 202 and septum wall 204 of catheter 200. With this arrangement, first and second mounting elements 116, 118 of bifurcated segment 114 of connector apparatus 1200 may be positioned within lumens 1406 of cover 1400 with septum wall 1408 within slot 124 defined between the first and second mounting elements 116,118 of the connector apparatus 1100. Bifurcated segment 114 secures cover 1400 in a similar manner to that described hereinabove in connection with catheter 200. For example, the irregularities of internal surfaces 126 deform septum wall 1404 creating a tortuous path, which substantially minimizes inadvertent removal of cover 1400 from connector apparatus 1200. Alternatively, internal surfaces 126 may be devoid of the irregularities and may establish an interference relation with septum wall 1408 of cover 1400. Lumens 1406 and septum wall 1408 may be dimensioned to correspond with the dimensioning of bifurcated segments 114 to achieve the releasable coupling relation. Leading section 1404 of cover 1400 is generally tapered or frusto-conical in configuration to facilitate passage of the cover 1400 and insertion stylet 1100 through the subcutaneous tissue.

In the mounted condition of cover 1400 on connector apparatus 1200, trailing section 1402 engages collar 1204 of connector body 102 to ensure that no tissue or fluids may enter beneath the cover 1400 and contact bifurcated segment 114. Trailing section 1402 may be at least partially positioned over collar 1204 of bifurcated segment 114.

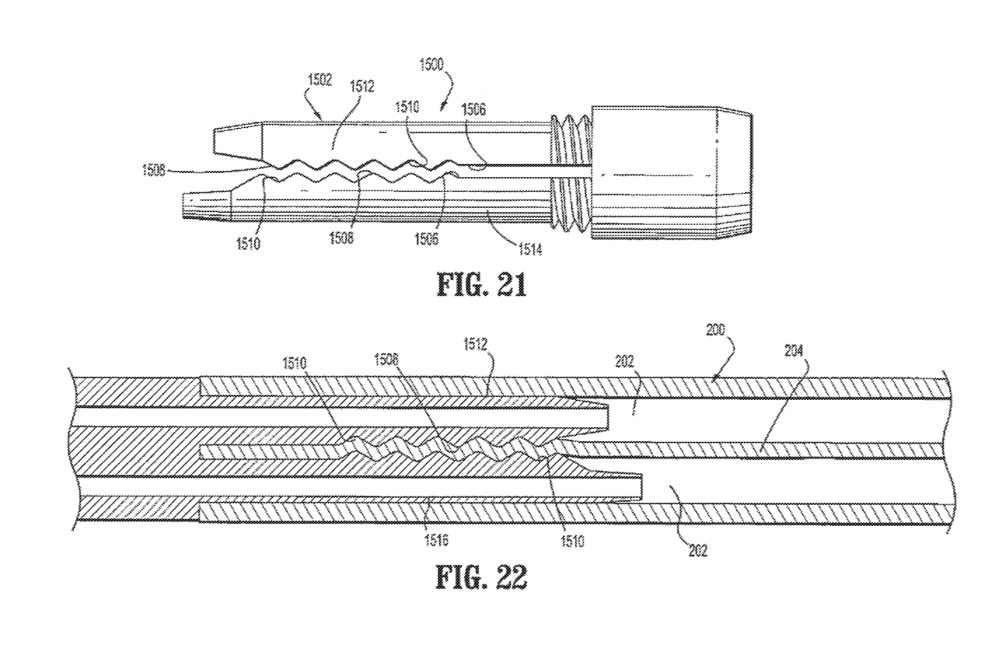

FIGS. 21-22 illustrate an alternate embodiment of connector apparatus. Connector apparatus 1500 includes bifurcated segment 1502 having irregular internal surfaces 1506 in the form of an undulating arrangement of peaks 1508 and valleys 1510 as shown. Peaks and valleys 1508, 1510 may be formed by the intersection of linear surface segments as shown, or, alternatively may have arcuate profiles to define a more rounded or a sinusoidal arrangement. Peaks and valleys 1508, 1510 of opposed internal surfaces 1506 are arranged whereby the peaks 1508 on the internal surface 1506 of one mounting element 1512 are diametrically opposed to corresponding valleys 1510 of the other mounting element 1514. Peaks and valleys 1508, 1510 cooperate to engage septum wall 204 of catheter 200 to define a tortuous path preventing inadvertent removal of the catheter 200 from connector apparatus 1500. Connector apparatus 1500 may be releasably couplable to insertion stylet as described in connection with the connector apparatus 100 of FIGS. 1-11, or may be monolithically or integrally formed with insertion stylet in a similar manner to that described in connection with connector apparatus 1200 of the embodiment of FIG. 12.

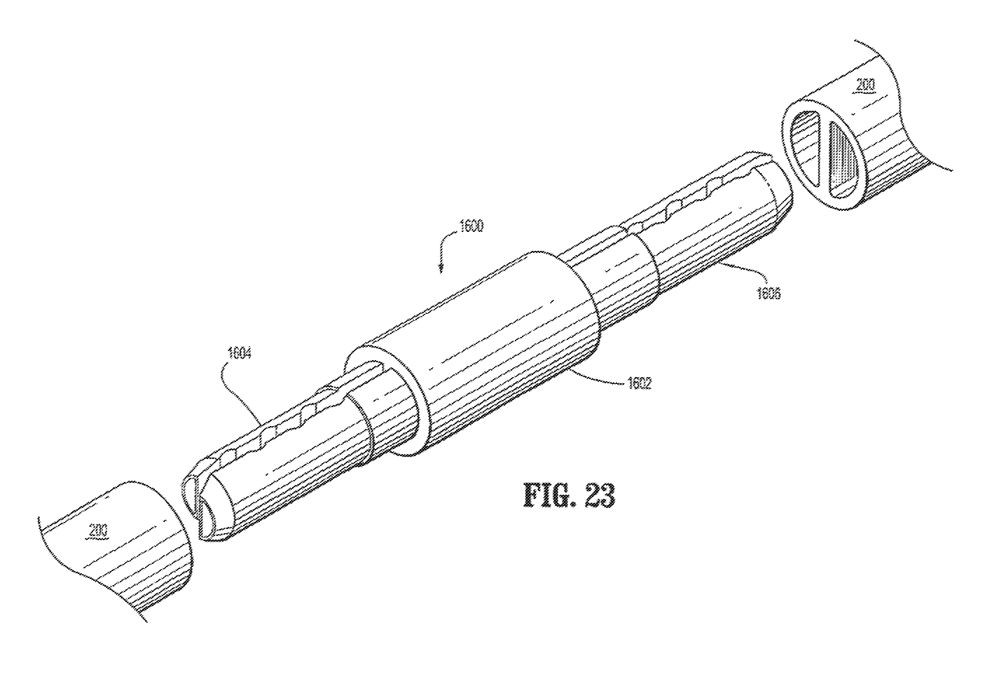

FIG. 23 illustrates another embodiment of the connector apparatus. Connector apparatus 1600 includes connector body 1602 having a pair of bifurcated segments 1604, 1606 disposed adjacent longitudinal ends of the connector body 1602. Each of the bifurcated segments 1604, 1606 may take the form of the bifurcated segments discussed in connection with the embodiment of FIG. 1. Connector apparatus 1600 permits the connection of first and second catheters 200, 200 in series, i.e., bifurcated segment 1604 may be connected to a first catheter 200 and bifurcated segment 1606 may be connected to a second catheter 200 thereby connecting the two catheters 200 in series. This arrangement permits coupling of various catheters 200 during a surgical procedure.

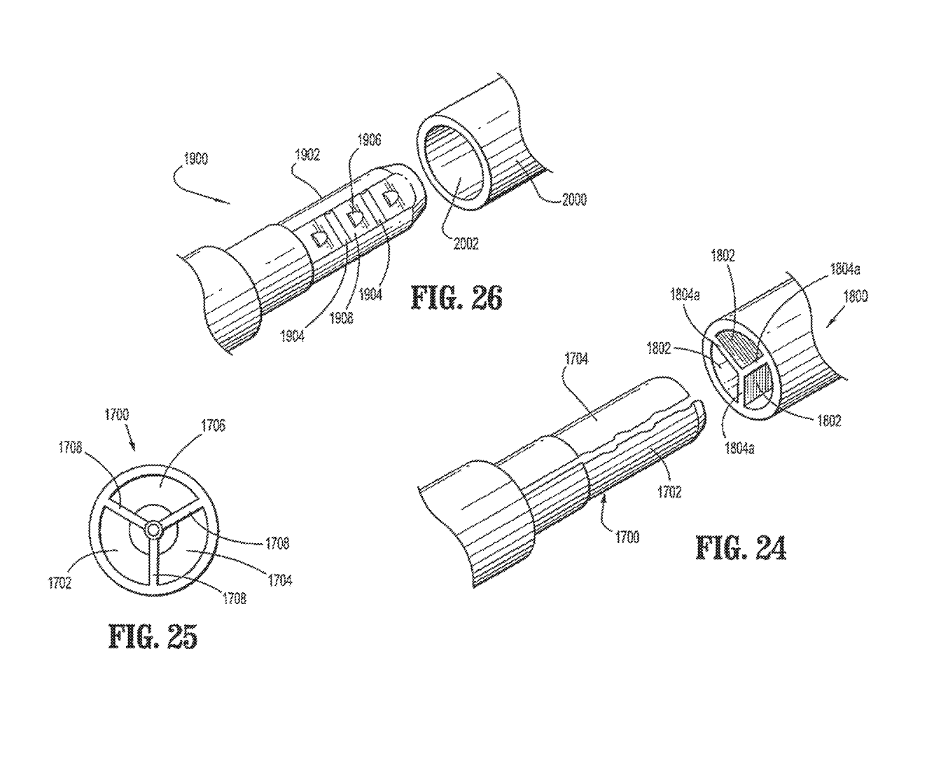

FIGS. 24-25 illustrate another embodiment of the connector apparatus. Connector apparatus 1700 includes first, second and third mounting segments 1702, 1704, 1706. Connector apparatus 1700 is adapted for use with triple lumen catheter 1800 whereby each mounting segment 1702, 1704, 1706 is received within a corresponding lumen 1802 of the catheter 1800. In one embodiment, adjacent mounting segments 1702, 1704, 1706 are separated by slots 1708 which receive a corresponding leg 1804a of the septum wall 1804. Legs 1804a may be secured within slots 1708 through engagement of inner surfaces 1710 of mounting segments 1702, 1704, 1706 in a similar manner to that described in connection with the prior embodiments of the connector apparatus.

As a further embodiment, with reference to FIG. 26, connector apparatus 1900 may include a single mounting component 1902. Single mounting component 1902 may be used with a single lumen catheter 2000. Single mounting component 1902 may have irregularities (e.g., concavities 1904 and/or projections 1906) which may be disposed on planar surface 1908 or may extend from the outer annular wall surface of single mounting segment if the mounting component 1902 is devoid of a planar surface 1908. Single mounting component 1902 may be devoid of irregularities. Single mounting component 1902 is adapted to engage an internal wall surface of single lumen catheter 2000 and is received within lumen 2002 of catheter 2000 in a similar manner to the embodiments discussed hereinabove to releasably or permanently secure connector apparatus 1900 to catheter 2000.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure. For example, the irregular surfaces of the internal surfaces of the connector apparatus may assume various other configurations such as incorporating rigid or flexible projections, nonplanar opposing internal surfaces, nonlinear gaps between the projections, mating nonplanar opposing surfaces, projections of the same or different configurations, curved opposed surfaces, coatings and various other shapes in configuration as appreciated by one skilled in the art. The connector apparatus may have three or more mounting elements for use with, e.g., a triple lumen catheter.

1-27. (canceled)

28. A connector for connecting a multiple lumen medical catheter to medical instrumentation, the connector comprising:
a first end securable to the medical catheter;
a second end securable to the medical instrumentation, a longitudinal axis extending from the first end to the second end; and
first and second D-shaped mounting elements adjacent the first end, the first and second mounting elements having opposed contacting surfaces defining a slot along the longitudinal axis, and the opposed contacting surfaces including irregular surface portions.

29. The connector of claim 28, wherein the first mounting element has a longitudinal length less than a longitudinal length of the second mounting element to define a staggered arrangement.

30. The connector of claim 28, wherein each of the first and second mounting elements has a tapered end surface.

31. The connector of claim 28, wherein the first and second mounting elements each have a smooth outer surface opposite the respective irregular surface portion.

32. The connector of claim 28, wherein the irregular surface portions of the respective first and second mounting elements each include an undulating surface portion.

33. The connector of claim 32, wherein the undulating surface portion includes a plurality of alternating apexes and recesses.

34. The connector of claim 33, wherein the apexes of one of the first and second mounting elements are in opposed relation to respective recesses of the other of the first and second mounting elements.

35. The connector of claim 28, wherein the irregular surface portions of the respective first and second mounting elements each include at least one projecting member having a leading surface extending at a first angle with respect to the longitudinal axis and a trailing surface extending at a second angle with respect to the longitudinal axis, and the second angle is not equal to the first angle.

36. The connector of claim 28, wherein a portion of the first and second mounting elements define a bifurcated collar adjacent the second end of the connector.

37. The connector of claim 36, wherein the bifurcated collar defines a cross-sectional dimension larger than corresponding cross-sectional dimensions of portions of the first and second mounting elements at the first end of the connector.

38. The connector of claim 37, wherein the opposed contacting surfaces are substantially planar along the bifurcated collar.

39. A method for implanting a catheter for use in association with a medical instrument, the method comprising:
inserting a first end of a catheter into the venous system of a patient through a first opening in tissue;
coupling a connector to a stylet;
positioning, at a second end of the catheter, first and second D-shaped mounting elements of the connector into corresponding D-shaped lumens of the catheter such that a septum wall of the catheter is disposed in a slot defined by opposing contact surfaces of the first and second mounting elements and irregular surface portions of the opposing contacting surfaces engage the septum wall;
advancing the stylet from the first opening to a second opening, away from the first opening, until the second end of the catheter is exposed at the second opening;
decoupling the connector from the second end of the catheter; and
connecting the second catheter end to a medical instrument.

40. The method of claim 39, wherein positioning the first and second D-shaped mounting elements of the connector into corresponding D-shaped lumens of the catheter comprises inserting the first D-shaped mounting element into a respective D-shaped lumen of the catheter in advance of inserting the second D-shaped mounting element into a respective D-shaped lumen.

41. The method of claim 39, wherein decoupling the connector from the second end of the catheter comprises exerting a linear force on the catheter.

42. The method of claim 39, wherein decoupling the connector from the second end of the catheter comprises cutting the catheter adjacent the connector.
